(19) United States
(12) Patent Application Publication
(10) Pub. No.: US 2018/0168670 A1
(43) Pub. Date: Jun. 21, 2018

(54) Instrument for Endoscopic Surgery

(71) Applicant: RZ Medizintechnik GmbH, Tuttlingen (DE)

(72) Inventor: Alexander Doppelstein, Tuttlingen (DE)

(21) Appl. No.: 15/578,740

(22) PCT Filed: Jun. 1, 2016
(86) PCT No.: PCT/EP16/62326

§ 371 Date: Dec. 1, 2017

(30) Foreign Application Priority Data
Jun. 6, 2015 (EP) 15170269.3

(51) Int. Cl. A61B 17/29

(52) U.S. Cl. CPC A61B 17/29; A61B 2017/2902; A61B 2090/0813; A61B 2017/294; A61B 2017/292; A61B 2017/2908

(57) ABSTRACT

The invention provides an instrument for endoscopic surgery comprising a shaft tube (10), a pull rod (12) which can move axially inside the shaft tube (10) and jaws (20) located at the distal end which are mounted on a single hinge pin (24) and can be pivoted towards each other. Relative axial motion of the shaft tube (10) and the pull rod (12) is converted into the pivot motion of the jaws (20) via contact eccentric to the hinge pin (24). The hinge pin (24) is mounted on the distal end of the pull rod (12). Each jaw (20) has a nib (34) which protrudes radially eccentric to the hinge pin (24). Each nib (34) engages in a cut-out (32) in a sheath (24, 124). The cut-outs (32) are covered by the shaft tube (10).

### TECHNICAL FIELD

The invention concerns an instrument for endoscopic surgery.

### BACKGROUND OF THE INVENTION

In endoscopic surgery, instruments are used which are often described as tubular shaft instruments. These instruments feature a shaft tube, inside which a pull rod can be moved in an axial manner. A handle located on the proximal end, e.g. a scissor handle, is used to move the pull rod in an axial manner inside the shaft tube, which opens and closes the jaws located on the distal end. The jaws may be designed differently for different applications, e.g. as forceps, scissors, coagulation instruments or similar.

In conventional instruments, the two jaws are usually pivot-mounted on a single hinge pin located on the distal end of the shaft tube. The jaws are designed as dual arm levers, with the pull rod engaging with the proximal lever arm of the jaws via hinged brackets. An example of this type of design is shown in DE 93 17 535 U1. This manner of manipulating the jaws via dual scissor hinges is cumbersome from a construction point of view. The scissor hinges are difficult to clean and sterilise. Additionally, the dual scissor hinges are freely accessible from the exterior and cannot be insulated for electro surgery applications.

An attempt was made to address these problems through an instrument of the type mentioned above, described in DE 10 2010 033 424 A1. In this instrument, the two jaws are pivot-mounted on a single hinge pin located on the distal end of the shaft tube.

The pull rod, which moves axially inside the shaft tube, engages with each jaw eccentrically to the hinge pin, which causes the axial motion of the pull rod to be converted into a pivot motion in the jaws. For this purpose, a pull bar is connected to the distal end of the pull rod; the angled arms of the pull bar, each in an eccentric recess, engage with the perimeter of the jaws. The multi-angled pull bar makes the device elaborate to manufacture and assemble.

An attempt was also made to address these problems in EP 0513471. The jaws were designed as being pivotable around a single hinge pin, each jaw having a nib protruding eccentrically into an opening. The design however had shortcomings in maintaining the jaws in the centre of the assembly and in allowing contamination through the openings. Some embodiments provided an external shank tube to eliminate openings, but these were of complex design.

GB 2,354,170 incorporated a tube and a two part keeper for an actuating member for its jaws. However, in GB 2,354,170, the jaw mechanism provided was complex, the actuating member having arms and extensions which required mating with a notch and recess. As a result, the precision and complexity required made assembly more costly and difficult.

In US 2006/161190, in attempting to solve the same problems, a tube and sheath were used but the jaw mechanism was once again too elaborate.

The invention is based on the aim of simplifying an endoscopic surgical instrument of the type mentioned above in terms of its manufacture and assembly, and improving it in terms of its applications.

### DISCLOSURE OF THE INVENTION

Accordingly, this invention provides an instrument for endoscopic surgery comprising:

a shaft tube,

a pull rod which can be moved in an axial manner inside the shaft tube; and

jaws located at the distal end which are mounted on a single hinge pin mounted on the distal end of the pull rod, the jaws being pivotable towards each other, whereby the relative axial motion of the shaft tube and pull rod is converted into the pivot motion of the jaws via contact eccentric to the hinge pin, each of the jaws having a nib which protrudes radially eccentric to the hinge pin, the nibs of the two jaws being diametrically opposed, each engaging in a cut-out,

characterised in that the cut-outs are made in a sheath which is secured coaxially inside the distal end of the shaft tube, the cut-outs being covered by the shaft tube.

In the instrument according to the invention, the two jaws are pivot-mounted on a single hinge pin which is mounted on the distal end of the pull rod. Preferably, the relative axial motion between the pull rod and the shaft tube is converted into the pivot motion by which the jaws open and close through a nib which protrudes radially from the perimeter of each jaw, eccentric to the pivot axis of the hinge pin, and which engages in a cut-out in the shaft tube. Each nib is held axially in its cut-out so that the axial motion of the hinge pin is converted through the pull rod into a rotary or pivot motion in the respective jaw.

The instrument is simple in terms of its manufacture and assembly. The jaws simply need to be mounted on the distal end of the pull rod by means of the hinge pin, without any need for additional components for the distal working end of the instrument. The pull rod with the jaws is inserted in the shaft tube so that the nibs on the jaws engage with their respective cut-outs. Only the working ends of the jaws protrude from the distal end of the shaft tube, while the actuating and pivot mechanism is located inside the distal end of the shaft tube. This allows the jaws to be easily cleaned and sterilised. The distal end of the shaft tube encompasses the swivel joint of the jaws and their actuation, making it possible to fit electrical insulation along the outer circumference of the shaft tube, up to the exposed working ends of the jaws at the distal end. If the instrument is designed for electro surgery purposes, e.g. as a coagulation instrument, the exposed electrode surface of the jaws is advantageously small so as to allow a defined and targeted application of high-frequency electric current to the tissue.

The cut-outs in which the nibs of the jaws engage in the sheath may take the form of apertures made in the wall of the sheath which is inserted coaxially and anchored inside the distal end of the shaft tube. The cut-outs formed as apertures in the wall of the sheath are thus covered and sealed at the outer circumference by the shaft tube. The shaft tube being completely closed along its outer circumference up to the distal end greatly reduces or prevents the penetration by contaminants into the pivot mechanism.

Furthermore, it enables the outer circumference of the shaft tube to be fully fitted with electrical insulation without interruption, up to the distal end of the shaft tube.

In a further preferred embodiment, either one end or both ends of the hinge pin on which the jaws are pivot-mounted may protrude beyond the outer circumference of the pull rod. This protruding end of the hinge pin engages in an axial slit made in the shaft tube, or preferably in the sheath. The hinge pin engaging in the axial slit prevents the pull rod, and therefore also the jaws mounted on the pull rod, from twisting in relation to the shaft tube. The positioning of the pivot plane of the jaws in relation to the instrument's proximal handle is thus clearly determined, which is of crucial importance for using the instrument.

It is preferred that a shaft retainer is fitted to the pull rod to serve as a limit stop to limit distal movement of the pull rod when it is pushed in a distal direction inside the sheath. The shaft retainer may take the form of a circlip.

In an alternate embodiment, the shaft retainer may be a limit pin through the pull rod, the limit pin cooperating with the sheath. In this embodiment, the limit pin may have one or each end protruding and accommodated in an aperture in the sheath to limit distal movement of the pull rod.

### BRIEF DESCRIPTION OF THE DRAWINGS

In the following, the invention is explained in greater detail with reference to non-limiting example embodiments illustrated in the drawings. It will be appreciated that not all drawings are drawn to the same scale. In the drawings:

FIG. 1 is a side view of a first embodiment of the instrument of the invention;

FIG. 2 is a side view of the instrument of FIG. 1, rotated through 90°;

FIG. 3 is an axial section of the instrument as per the cutting line A-A in FIG. 2;

FIG. 4 shows an enlarged detail view as per area B in FIG. 3;

FIG. 5 shows a side view of the distal end of the instrument with the shaft tube removed, the jaws in FIG. 5 being spread further apart than in FIG. 4;

FIG. 6 is a perspective view of FIG. 5;

FIG. 7 shows an isolated perspective view of the sheath in the first embodiment;

FIG. 8 is a side perspective view of the distal end of a second embodiment of the instrument of the invention, with the shaft tube removed;

FIG. 9 shows a similar view to that of FIG. 8, with the sheath removed; and

FIG. 10 is a side elevation of the distal end of the second embodiment, with the sheath in place, showing separately the shaft tube and an insulating sleeve.

### DETAILED DESCRIPTION OF THE DRAWINGS

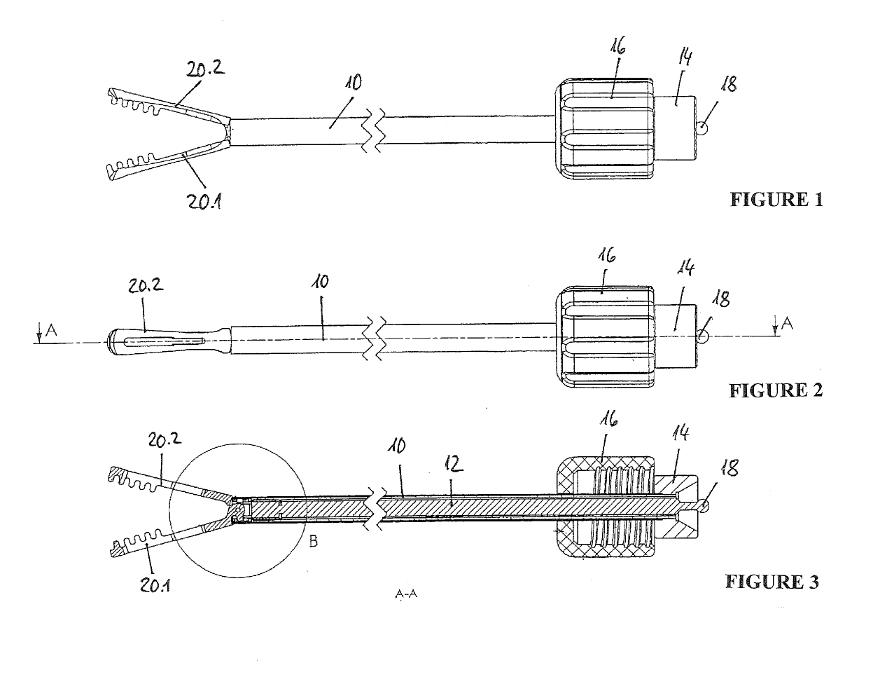

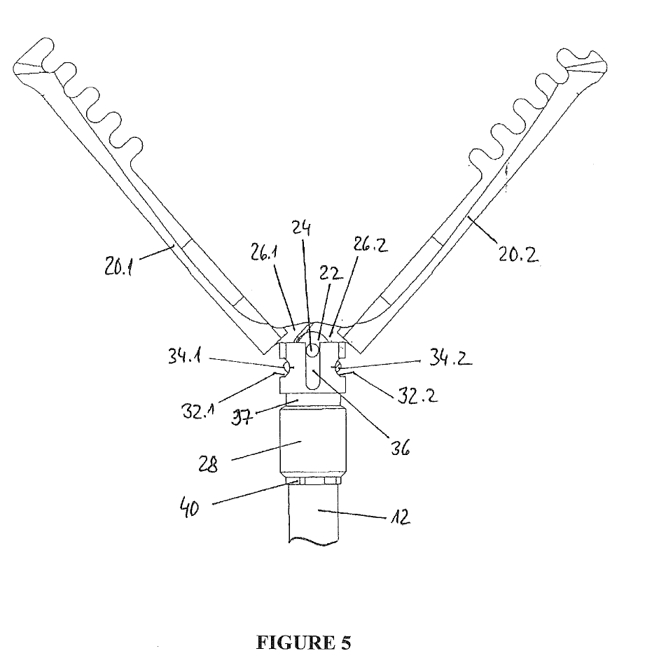

The first embodiment of the instrument for endoscopic surgery according to the invention is shown in FIGS. 1 to 7. It features a rigid shaft tube 10 in which a pull rod 12 can be moved in an axial manner. There is a handle located on the proximal end of the instrument; this is a known feature and is not included in the drawing. The handle, which may for example be designed as a scissor handle, features a fixed handle part, as well as a movable handle part which actuates the instrument. The proximal end of the shaft tube 10 is connected to the fixed part of the handle via a stop bush 14 and union nut 16. The proximal end of the pull rod 12 features a spherical head 18 which is seated in the moving part of the handle, so that manipulating the movable part of the handle causes the pull rod 12 to be move axially inside the shaft tube 10.

The distal end of the instrument features two jaws, 20.1 and 20.2, which can be moved towards each other in a closing movement or apart from each other in an opening movement by the axial displacement of the pull rod 12. The closing movement results from active actuation of the instrument, and requires a greater force to act on the jaws 20. For this reason, it is preferable to actuate the closing movement through pulling force applied to the pull rod 12 in a proximal direction, which is why this component is called a “pull rod”.

Pushing the pull rod 12 in a distal direction generates a distal thrust which causes the jaws 20 to move apart. The jaws 20 may be designed in a known manner, depending on the intended use of the instrument. In the illustrated example embodiment, the jaws 20 are designed as grasping forceps. The jaws 20 may equally be designed as scissors, as a clamp or as a coagulation clamp.

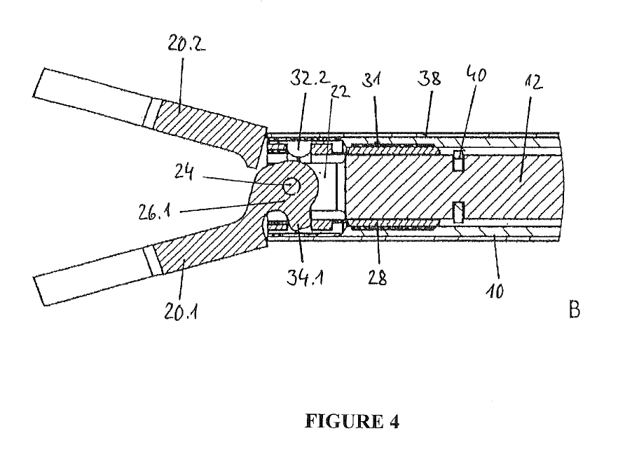

As shown in FIG. 4, the distal end of the pull rod 12 is designed as a bracket 22 which opens in the distal direction. The bracket 22 has a bore, running perpendicular to the axis of the pull rod 12, into which a hinge pin 24 is inserted. Only the proximal parts of jaws 20.1 and 20.2 are shown. The proximal ends of the jaws 20.1 and 20.2 are designed as flat discs 26.1 and 26.2. The discs 26.1 and 26.2 are inserted into the bracket 22 side by side with the flat sides contiguous. The hinge pin 24 passes through the centre of the discs 26.1 and 26.2, so that the jaws 20.1 and 20.2 are pivot-mounted on the hinge pin 24 on the same plane as the discs 26.

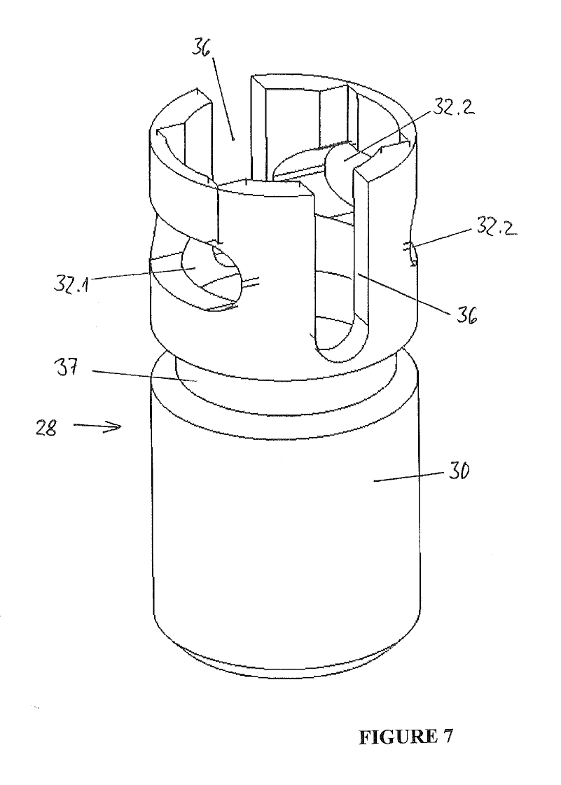

On the distal end of the pull rod 12, there is a sheath 28, which can slide freely in an axial manner; the sheath 28 for this embodiment is illustrated in detail in FIG. 7. The sheath 28 is inserted coaxially into the distal end of the shaft tube 10, and is secured axially and against rotation inside the shaft tube 10. For this purpose, the proximal end portion 30 of the sheath 28 is provided with an external thread 31 which screws into a corresponding internal thread (not labelled) in the shaft tube 10. Other alternatives for securing the sheath 28 inside the shaft tube 10 are also possible. For example, the end portion 30 (FIG. 7) of the sheath 28 can be secured inside the shaft tube 10 by means of a bayonet joint, glue, pressing or by other means.

The distal end portion of the sheath 28 has two cut-outs, 32.1 and 32.2, which are designed as diametrically opposing apertures in the wall of the sheath 28. Each of the discs, 26.1 and 26.2, on the jaws, 20.1 and 20.2, features a moulded nib, 34.1 and 34.2. Each nib, 34.1 and 34.2, is moulded on the outer circumference of the discs, 26.1 and 26.2, eccentric to the hinge pin 24, and protrudes from the circumference of the discs 26.1 and 26.2 in an essentially radial manner. The nibs, 34.1 and 34.2, are located on the circumference of the discs, 26.1 and 26.2, such that that they are diametrically opposed to each other and protrude outwards from the hinge pin 24. The nibs 34.1 and 34.2 on each jaw 20.1 and 20.2 engage in the cut-outs 32.1 and 32.2 in the sheath 28; this is most clearly illustrated in FIG. 4.

If, while the instrument is in use, the pull rod 12 is pushed forward axially in the shaft tube in the distal direction by means of the handle which is not illustrated, this moves the hinge pin 24 on which the jaws 20.1 and 20.2 are mounted in a distal direction in relation to the shaft tube 10 and to the sheath 28 which is fitted securely inside the shaft tube 10. At the same time, the nibs 34.1 and 34.2 on the jaws 20.1 and 20.2 are secured axially in the corresponding cut-outs 32.1 and 32.2 in the sheath 28. Because of the eccentric position of the nibs 34.1 and 34.2, this causes the jaws 20.1 and 20.2 to pivot around the hinge pin 24. As illustrated in FIG. 4, the jaw 20.1 is pivoted anticlockwise, owing to nib 34.1 engaging in cut-out 32.1, while jaw 20.2 is pivoted clockwise, owing to nib 34.2 engaging in cut-out 32.2. Pushing the pull rod 12 forwards in the distal direction thus causes the jaws 20.1 and 20.2 to open.

Conversely, if the pull rod 12 is pulled in the proximal direction inside the shaft tube 10 by means of the handle, which is not illustrated, this moves the hinge pin 24 with the jaws 20.1 and 20.2 into the sheath 28. The nibs 34.1 and 34.2 engaging in the axially fixed cut-outs 32.1 and 32.2 are pivoted in opposing directions around the hinge pin 24, so that jaw 20.1 illustrated in FIG. 4 is pivoted clockwise and jaw 20.2 is pivoted anticlockwise; this causes the mouth formed by these jaws to close.

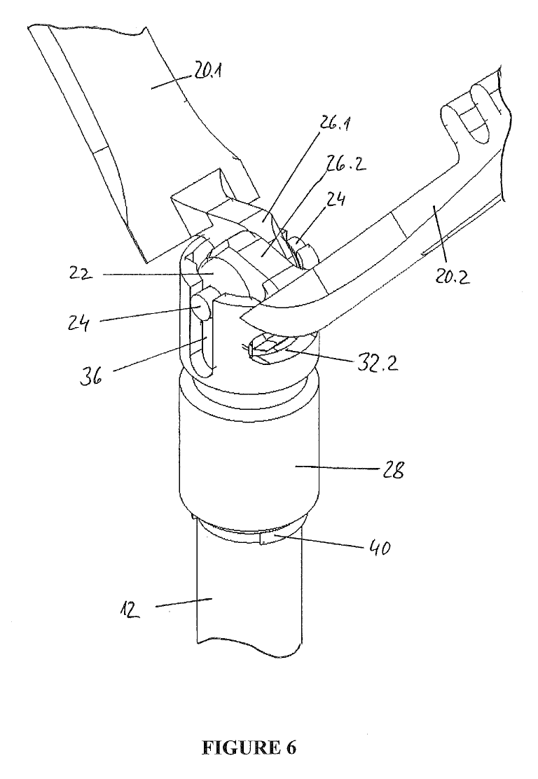

The distal end of the sheath 28 features one, or, as illustrated in the first embodiment, two diametrically opposed axial slits 36 which are open at the distal edge of the sheath 28. The slits 36 are each offset from the cut-outs 32 by 90°. As illustrated most clearly in FIG. 6, one end or preferably both ends of the hinge pin 24 protrude beyond the outer circumference of the pull rod 12. This protruding end of the hinge pin 24 engages radially in the slit 36. The axial length of the slit 36 enables the pull rod 12 and hinge pin 24 to move axially inside the sheath 28 which is secured inside the shaft tube 10. The engaging of the hinge pin 24 in the slit 36 prevents the pull rod 12 from twisting while being moved inside the shaft tube 10. As a result, the pivot plane of the jaws 20 mounted on the pull rod 12 is set in relation to the shaft tube 10 and the handle connected to the shaft tube, and cannot be twisted.

The first embodiment of the instrument is assembled as follows:

First, jaws 20.1 and 20.2 are inserted into the bracket 22 of the pull rod 12, and mounted on the pull rod by means of the hinge pin 24. Then the sheath 28 is slid onto the pull rod 12 and pushed forwards in the distal direction towards the jaws 20. During this process, the jaws 20 should be open wide enough that the nibs 34.1 and 34.2 are fully inserted into the openings in the inner profile of the sheath 28. The sheath 28 can thus be slid over the inwardly pivoted nibs 34. Once the sheath 28 with the cut-outs 32.1 and 32.2 is aligned axially with the nibs 34.1 and 34.2, the jaws 20.1 and 20.2 are pivoted into the closed position so that the nibs 34.1 and 34.2 engage in the corresponding cut-outs 32.1 and 32.2 in the sheath 28. Then the pull rod 12 with the sheath 28 fitted is inserted into the shaft tube 10. The sheath 28 is secured axially on the pull rod 12 by the nibs 34 engaging in the cut-outs 32, while the hinge pin 24 engaging in the slits 36 prevents the sheath 28 from twisting on the pull rod 12. Using the pull rod 12, the threaded 31 end portion 30 of the sheath 28 can thus be screwed into the internal thread of the shaft tube 10 so as to anchor the sheath 28 inside the shaft tube 10. A recessed part 37 (FIG. 7) delimits the distal end of the thread on the end portion 30.

Once the sheath 28 is fully inserted into the distal end of the shaft tube 10, the shaft tube 10 overlaps the apertures created by the cut-outs 32 and the slits 36 so that they are fully covered and sealed at the outer circumference of the shaft tube 10. The shaft tube 10 is therefore fully closed and without apertures right up to the jaws 20 at the distal end. Preferentially, the outer lateral surface of the shaft tube 10 can be fitted with electrical insulation 38 (FIG. 4), which likewise is uninterrupted right up to the jaws 20 at the distal end. This makes the instrument particularly suitable for electro surgery applications.

Before the pull rod 12 with the sheath 28 fitted is inserted into the shaft tube 10, a shaft retainer 40 is preferentially inserted proximally behind the sheath 28 into a recess on the pull rod 12. The shaft retainer 40 can move freely in an axial manner inside the shaft tube 10; however, inside the sheath 28 anchored in the shaft tube 10 it forms a limit stop which limits the movement of the pull rod 12 when pushed forward in the distal direction. In this way, the shaft retainer 40 limits the forward movement in the distal direction of the pull rod 12. This prevents the pull rod 12 from being pushed so far forward that the jaws 20.1 and 20.2 are opened to the maximum extent, causing the nibs 34.1 and 34.2 to pivot inwards and come out of the cut-outs 32.1 and 32.2.

In the first embodiment, the shaft retainer 40 is in the form of a circlip.

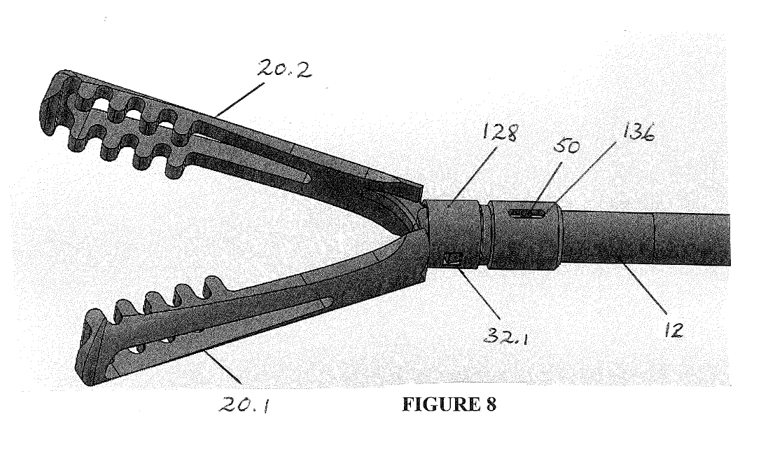

Turning now to the second embodiment of the instrument of the invention, as illustrated in FIGS. 8 to 10, this embodiment works in much the same way as the first embodiment, except for the arrangement concerning the sheath and the shaft retainer. The parts which are the same as those in the first embodiment are referred to using the same labels.

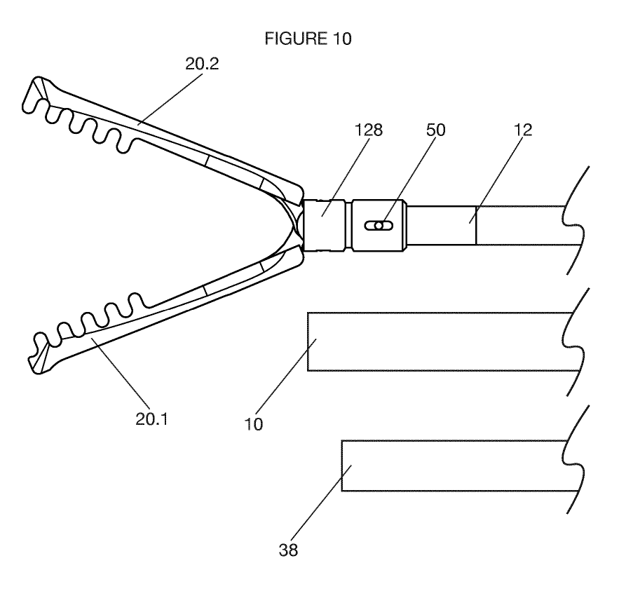

As shown in FIG. 8, on the distal end of the pull rod 12 there is a sheath 128 mounted on the pull rod 12. The sheath 128 is inserted coaxially into the distal end of the shaft tube 10 (shown in FIG. 10) and is secured axially and against rotation inside the shaft tube 10 as explained further below.

The distal end portion of the sheath 128 has two cut-outs, 32.1 and 32.2 (32.2 is not visible), which are the same as those in the previous embodiment and which are designed as diametrically opposing apertures in the wall of the sheath 128. Cut-outs 32.1 and 32.2 accommodate moulded nibs, 34.1 and 34.2 as described for the previous embodiment.

Jaws 20.1 and 20.2 operate as described for the previous embodiment.

In the second embodiment, the distal end of the sheath 128 features two diametrically opposed axial apertures 136 which are not open at the distal edge of the sheath 128 but are fully within the wall of the sheath 128. (Only one aperture 136 is visible in FIGS. 8 to 10.) The apertures 136 are each offset from the cut-outs 32 by 90°.

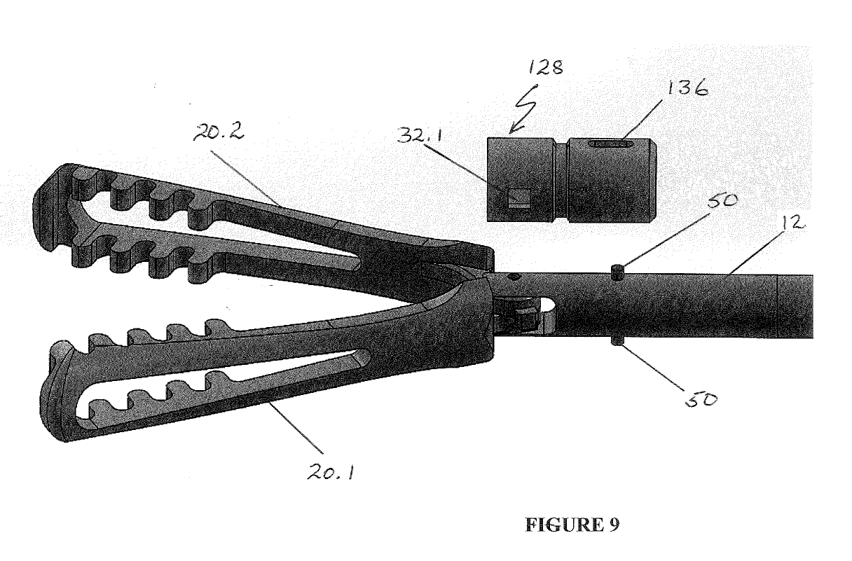

A fixed limit pin 50 has each end protruding as shown in FIG. 9. Pin 50 may be fixed by laser welding or any other suitable method. When sheath 128 is assembled, the ends of pin 50 are accommodated in apertures 136 (see FIG. 8). The engaging of the pin 50 in the aperture 136 prevents the pull rod 12 from twisting relative to sheath 128 while being moved inside the shaft tube 10. Sheath 128 is fixed to shaft tube 10, such as by screw threads or adhesive or by a bayonet fitting cooperating with apertures in shaft tube 10. As a result, the pivot plane of the jaws 20 mounted on the pull rod 12 is set in relation to the shaft tube 10 and the handle connected to the shaft tube 10, and cannot be twisted.

Pin 50 also prevents rotation of sheath 128 within shaft tube 10.

Movement of sheath 128 distally and proximally is limited by the length of apertures 136 and engagement by the protruding ends of the pin 50. At the same time, pin 50 in apertures 136 forms a limit stop which limits the movement of the pull rod 12 when pushed forward in the distal direction. In this way, the pin 50 limits the forward movement in the distal direction of the pull rod 12. This prevents the pull rod 12 from being pushed so far forward that the jaws 20.1 and 20.2 are opened to the maximum extent, causing the nibs 34.1 and 34.2 to pivot inwards and come out of the cut-outs 32.1 and 32.2, so that the distal movement of the pull rod 12 with the jaws 20 inside the shaft tube 10 is no longer limited.

Although pin 50 is shown with both ends protruding (FIG. 9), requiring diametrically opposed apertures 136 in sheath 124 (one aperture 136 being shown in FIG. 8, the other aperture not visible), it is within the scope of the invention that pin 50 may instead protrude at one end only, in which case a single aperture 136 would be required to accommodate that end.

The second embodiment of the instrument is assembled as follows:

First, jaws 20.1 and 20.2 are inserted into the bracket 22 of the pull rod 12, and mounted on the pull rod by means of the hinge pin 24. Then the sheath 128 is slid onto the pull rod 12. During this process, the jaws 20 should be open wide enough that the nibs 34.1 and 34.2 are fully inserted into the openings in the inner profile of the sheath 128. The sheath 128 can thus be slid over the inwardly pivoted nibs 34. Once the sheath 128 with the cut-outs 32.1 and 32.2 is aligned axially with the nibs 34.1 and 34.2, the jaws 20.1 and 20.2 are pivoted into the closed position so that the nibs 34.1 and 34.2 engage in the corresponding cut-outs 32.1 and 32.2 in the sheath 128.

Limit pin 50 is fixed to pull rod 12, such as by laser welding, with one end protruding into and being received in aperture 136 of sheath 124. Alternately, each end of limit pin 50 protrudes into and is received by an aperture 136.

The limit pin 50 engaging in the aperture or apertures 136 prevents the sheath 128 (and pull rod 12) from twisting.

Shaft tube 10 is then fitted and fixed to sheath 128.

Once the sheath 128 is fully inserted into the distal end of the shaft tube 10, the shaft tube 10 overlaps the apertures created by the cut-outs 32 and the aperture/s 136 so that they are fully covered and sealed at the outer circumference of the shaft tube 10. The shaft tube 10 is therefore fully closed and without apertures right up to the jaws 20 at the distal end. Preferentially, the outer lateral surface of the shaft tube 10 can be fitted with electrical insulation 38 (FIG. 10), which likewise is uninterrupted right up to the jaws 20 at the distal end. This makes the instrument particularly suitable for electro surgery applications.

1. Instrument for endoscopic surgery comprising:
a shaft tube (10),
a pull rod (12) which can be moved in an axial manner inside the shaft tube (10); and
jaws (20.1, 20.2) located at the distal end which are mounted on a single hinge pin (24) mounted on the distal end of the pull rod (12), the jaws (20) being pivotable towards each other, whereby the relative axial motion of the shaft tube (10) and pull rod (12) is converted into the pivot motion of the jaws (20) via contact eccentric to the hinge pin (24), each of the jaws (20) having a nib (34) which protrudes radially eccentric to the hinge pin (24), the nibs (34.1, 34.2) of the two jaws (20) being diametrically opposed, each engaging in a cut-out (32.1, 32.2),
characterised in that the cut-outs (32.1, 32.2) are made in a sheath (28, 128) which is secured coaxially inside the distal end of the shaft tube (10), the cut-outs (32) being covered by the shaft tube (10).

2. Instrument as in claim 1,
characterised in that a shaft retainer (40, 50) fitted to the pull rod (12) serves as a limit stop to limit distal movement of the pull rod (12) when it is pushed in a distal direction inside the sheath (28).

3. Instrument as in claim 2,
characterised in that the shaft retainer (40) is a circlip.

4. Instrument as in claim 2
characterised in that the shaft retainer is a limit pin (50) through the pull rod (12), the pin (50) having at least one protruding end accommodated in an aperture (136) in the sheath (128), to limit distal movement of the pull rod (12).

5. Instrument as in claim 4,
characterised in that the limit pin (50) has each end protruding from the pull rod (12) and accommodated in an aperture (136) in the sheath (128).

6. Instrument as in of claim 1,
characterised in that the sheath (28, 128) is screwed, pressed or glued into the shaft tube (10).

7. Instrument as in claim 6,
characterised in that the sheath (28, 128) has an external thread which is screwed into an internal thread in the shaft tube (10), whereby the thread determines the defined axial position of the sheath(28, 128) inside the shaft tube (10).

8. Instrument as in claim 1,
characterised in that at least one end of the hinge pin (24) protrudes beyond the circumference of the pull rod (12) and engages in an axial slit (36, 136) in the shaft tube (10) or the sheath (28), whereby the pull rod (12) with the jaws (20.1, 20.2) is prevented from twisting as it moves inside the shaft tube (10).

9. Instrument as in claim 8,
characterised in that at least one axial slit (36, 136) is formed in the sheath (28, 128), and is covered by the shaft tube (10).

10. Instrument as claim 1,
characterised in that the outer circumference of the shaft tube (10) is fitted with electrical insulation (38), up to the distal end adjacent to the jaws (20.1, 20.2).
